(19) United States
(12) Patent Application Publication
(10) Pub. No.: US 2025/0143246 A1
(43) Pub. Date: May 8, 2025

(54) TIPBURN-RESISTANT LETTUCE PLANT, PRODUCTION METHOD FOR TIPBURN-RESISTANT LETTUCE PLANT, AND METHOD FOR GIVING LETTUCE PLANT TIPBURN-RESISTANCE

(71) Applicants: YOSHINOYA HOLDINGS CO., LTD., Chuo-ku, Tokyo (JP); LEAFLAB CO., LTD., Kashiwa-shi, Chiba (JP)

(72) Inventors: Satoru KIMURA, Chuo-ku, Tokyo (JP); Tomoko TSUJI, Chuo-ku, Tokyo (JP); Toru MARUO, Kashiwa-shi, Chiba (JP); Konomi YOKOTA, Kashiwa-shi, Chiba (JP); Ana Paula ITO, Kashiwa-shi, Chiba (JP); Kosuke AMENOMORI, Kashiwa-shi, Chiba (JP); Asuya INOUE, Kashiwa-shi, Chiba (JP)

(21) Appl. No.: 18/838,254

(22) PCT Filed: Feb. 3, 2023
(86) PCT No.: PCT/JP2023/003551

§ 371 Date: Aug. 14, 2024

(30) Foreign Application Priority Data
Feb. 17, 2022 (JP) 2022-022864

(51) Int. Cl. A01H 6/14; A01H 5/12; C12N 15/82; C12Q 1/6895

(52) U.S. Cl. CPC A01H 6/1472; A01H 5/12; C12N 15/8261; C12Q 1/6895; C12Q 2600/13; C12Q 2600/156

(57) ABSTRACT

Provided are a novel tipburn-resistance marker for lettuce plant, a tipburn-resistant lettuce plant, and a production method for the tipburn-resistant lettuce plant that uses the tipburn-resistance marker. Disclosed is a tipburn-resistant lettuce plant that includes a tipburn-resistance gene locus on the 4th chromosome, the tipburn-resistance gene locus being specified by at least one SNP marker selected from the group that consists of NC_056626.1-271576390, NC_056626.274188377, NC_056626.1-274188380, and NC_056626.274188382.

### TECHNICAL FIELD

The present invention relates to a tipburn-resistant lettuce plant, a production method for a tipburn-resistant lettuce plant, and a method for giving a lettuce plant tipburn-resistance.

### BACKGROUND ART

The tipburn symptom found in the lettuce plant cultivation is the physiological disorders which occur at the immature inner leaves as calcium deficiency by the short supply of calcium ion that is the cell wall constituent to the growth rate of the lettuce individual leaf. The supply of calcium is determined by the absorption from the roots and the move of calcium absorbed in the body. The occurrence of the tipburn is also affected by the factor related to the growth rate of the plant in addition to these two factors related to the supply of calcium. Additionally, because the move of calcium absorbed in the body is largely affected by the change of the leaf area with the growth, the factor analysis is extremely complicated. Because the cultivation environment is unstable in the open-field cultivation condition and the greenhouse cultivation condition so far, the reproducibility of the occurrence is extremely low. Thus, the breeding of the resistant cultivars, the cultivative control of tipburn are not sufficient. Because the parts where the tipburn occur become burned brown, which is the symptom of the tipburn and the irreversible necrosis of the parts is caused, the growth is also delayed when the damage is large. It is known that the tipburn occur especially by the sharp increase in the temperature and the dryness in the open-field cultivation and the greenhouse cultivation (Non-Patent Document 1).

In the plant factories with artificial light which are increasing recently, because the growth rate is remarkably faster than in the general open-field cultivation, the risk of the occurrence of the tipburn become extremely high. The decrease of the yield and the increase of the labor cost due to the slow growth, and reduced yields and increased labor costs due to trimming the part damaged by tipburn, are becoming problems. Under the circumstances the growth rate is restrained on purpose by setting the illumination intensity low and the Lighting hours per day short to inhibit the occurrence of the tipburn. Additionally, the countermeasures such as harvesting small plant before the tipburn occur are conducted. Consequently, the economical productivity of the plant factory is largely restrained (Non-Patent Document 2, Non-Patent Document 3).

From the above reason, the lettuce plant breed exhibiting sufficient resistance against the tipburn have not been raised so far. The development of the breed exhibiting high resistance against the tipburn and growing faster at the same time is required especially for the plant factory with artificial light where the growth rate is high (Non-Patent Document 4).

The lettuce plant breed exhibiting sufficient resistance against the tipburn have not been raised so far. The development of the breed exhibiting high resistance against the tipburn is required especially for the plant factory with artificial light. The tipburn-resistance evaluation indexes (The tipburn-resistance is evaluated on a scale of one to ten. The tipburn-resistance evaluation index is described as “TB index”.) of the existing breeds in the plant factory with artificial light where the tipburn occur remarkably are shown in Table.1. The larger the number of the resistance evaluation index, the higher the resistance.

TABLE 1

| | | Variety | | |
|---|---|---|---|---|
| | The lettuce | registration | Product lot | TB |
| Seller | plant breed | number | number | index |
| TAKII | Greeen Jacket | TLE460 | V082 | 2.3 |
| TAKII | Summer Rouge | TLE416 | H063 | 2.5 |
| SAKATA SEED | Rivergreen | 10378 | | 1.7 |
| SAKATA SEED | Intercept | SAKLET001 | HAV0604 | 1.8 |
| The Yokohama Nursery | Notip | | 8TA83 | 3.2 |
| The Yokohama Nursery | Handsomegreen | XXL-5 | 6BNZ7 | 2.3 |
| The Yokohama Nursery | Marino | | 8AEW8 | 3.1 |
| Vilmorin Mikado | Green Impulse | | N95501 | 3.2 |
| Vilmorin Mikado | Early Impules | | P08515 | 3.4 |
| TSURUTA SEED | Notch Greengrass | Notch Greengrass | | 3.5 |
| TSURUTA SEED | Crunch | | 1228-011 | 3.8 |
| SNOW BRAND SEED | Frill Ice | Frill Ice | C11294108 | 1.8 |
| Rijk Zwan | Lalique | LALIQUE | 102033473 | 3.8 |
| Syngenta | LE2005 | | | 1.5 |
| Syngenta | Smashaki | CRISPITA II | R0107 | 3.8 |
| Syngenta | SYL648 | | 14038346 | 2.3 |
| Nakahara Seed | L-121 | | 2068802 | 3.5 |
| Nakahara Seed | L-122 | | 2562302 | 3.7 |
| Nakahara Seed | Fancy Green | | 1809204 | 3.2 |

TAKII: TAKII & CO., LTD
SAKATA SEED: SAKATA SEED CORPORATION
The Yokohama Nursery: The Yokohama Nursery Co., Ltd.
Vilmorin Mikado: Vilmorin Mikado Co., Ltd.
TSURUTA SEED: TSURUTA SEED Co., Ltd.
SNOW BRAND SEED: SNOW BRAND SEED Co., Ltd.
Rijk Zwaan: Rijk Zwaan Zaadteelt en Zaadhandel B. V.
Syngenta: Syngenta Japan Co., Ltd.
Nakahara Seed: Nakahara Seed Product Co., Ltd.

### CITATION LIST

### Non-Patent Document

Non-Patent Document 1: Ikeda, Hideo. 1990. Nogyo Gijutu Taikei [Series of Agricultural Technology]. Soil fertilization series [Part of Soil Fertilization]. vol. 4. addenda no. 1: 397-400.
Non-Patent Document 2: Maruo, Toru. 2019. Shokubutukojo niokeru Saibaigijutu oyobi Hinshukaihatu [Cultural Technology and Development of Breed in Plant Factory]. Yasai Joho [Information of Vegetables]. November 1990: 39-44. Noutikusanngyo Sinko Kiko.
Non-Patent Document 3: Kazai, T., G. Niu, and M. Takagaki. 2016. Plant factory. Academic press, London, UK.
Non-Patent Document 4: Maruo, Toru. 2019. Shokubutukojo muke Hinshukaihatu no Genzai [Current Situation of Development of Breed for Plant Factory]. Noko to Engei [Cultivation and Horticulture]Summer issue 2019: 30-34. Seibundosinkosha.

### SUMMARY OF INVENTION

### Technical Problem

The purpose of the present invention is to provide a novel tipburn-resistance marker for lettuce plant, a tipburn-resistant lettuce plant, a production method for the tipburn-resistant lettuce plant that uses the tipburn-resistance marker, and a method for giving lettuce plant tipburn-resistance.

### Solution to Problem

By the earnest research, the present inventors found that the lettuce plant including the tipburn-resistance gene locus (hereinafter also referred to as “resistance gene locus”) on the 4th chromosome as the tipburn-resistance marker (hereinafter also referred to as “resistance marker”) has tipburn-resistance so as to finish the present invention.

That is, the present invention relates to:

[1]A tipburn-resistant lettuce plant comprising a tipburn-resistance gene locus on a 4th chromosome,
wherein the tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.
   [2] The tipburn-resistant lettuce plant according to [1], wherein the tipburn-resistant lettuce plant is a lettuce plant specified by FERM AP-22442 or a progeny line thereof.
   [3] The tipburn-resistant lettuce plant according to [1] or [2], wherein the tipburn-resistant lettuce plant is a plant body or a part thereof.
   [4] The tipburn-resistant lettuce plant according to any one of [1] or [2], wherein the tipburn-resistant lettuce plant is a seed.
   [5]A method for producing a tipburn-resistant lettuce plant characterized by comprising following steps (A) and (B):
(A) a step of crossing the tipburn-resistant lettuce plant according to any one of Claims 1 to 4 with other lettuce plant,
(B) a step of picking up a tipburn-resistant lettuce plant from lettuce plants obtained in the step (A) or a progeny line thereof.
   [6] The method for producing a tipburn-resistant lettuce plant according to [5], comprising a following step (C) before the step (A):
(C) a step of picking up the tipburn-resistant lettuce plant according to any one of [1] to [4] from lettuce plants that have been tested.
   [7] The method for producing a tipburn-resistant lettuce plant according to [6], wherein the picking up in the step (C) is picking up of the tipburn-resistant lettuce plant including the tipburn-resistance gene locus on the 4th chromosome specified by at least one polynucleotide of followings (a), (b), (c), and (d),
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.
   [8]A method for giving lettuce plant tipburn-resistance comprising a step of introducing a tipburn-resistance gene locus on the 4th chromosome into a lettuce plant and that a tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.
   [9] The method for giving lettuce plant tipburn-resistance according to [8], wherein the tipburn-resistance gene locus is a tipburn-resistance gene locus specified by at least one of the polynucleotides of (a), (b), (c), and (d) of a lettuce plant specified by FERM AP-22442.
   [10]A method for screening a tipburn-resistant lettuce plant is comprising a step of picking up a lettuce plant including a tipburn-resistance gene locus on a 4th chromosome from lettuce plants that have been tested as parents for producing a tipburn-resistant lettuce plant by crossing and that the tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.

### Effects of the Invention

By using the tipburn-resistance marker found in the present invention, for example, the tipburn-resistant lettuce plant can be screened easily. The tipburn-resistant lettuce plant of the present invention can exhibit, for example, tipburn resistance because of including the resistance gene locus. Because of this, by using the tipburn-resistant lettuce plant of the present invention, the delay of the growth due to tipburn decrease and the trimming of the part damaged by tipburn become unnecessary. Thus the decrease of the yield or the problems of the work labor and the cost can be avoided.

### BRIEF DESCRIPTION OF DRAWINGS

FIG. 1 shows the schematic diagram showing the relative positions on which SNP (single nucleotide polymorphism) and the like are mounted on the 4th chromosome.

FIG. 2 shows the photographs showing the degree of the tipburn damage of the lettuce plants in Example 1

### DESCRIPTION OF EMBODIMENTS

### 1. Tipburn-Resistance Marker for Lettuce Plants

The tipburn-resistance marker for lettuce plants is characterized by including the tipburn-resistance gene locus on the 4th chromosome as described above, and the other features and the conditions are not particularly limited.

In the present specification, “lettuce plant” is the plant classified as the genus Lactuca (Lactuca sativa).

In the present specification, “tipburn” is also described as “Tip Burn”.

In the present invention, “tipburn resistance” is also referred to as, for example, “tipburn tolerance”.

The tipburn resistance means, for example, the ability to inhibit or to prevent the occurrence and the progress of the damage due to the occurrence of the tipburn, and the tipburn resistance may be specifically, for example, any one of the non-occurrence of the damage, the stop of the progress of the occurring damage, the prevention (also referred to as “inhibition”) of the progress of the occurring damage and the like.

Note that as shown in FIG. 2, the symptom of the black discoloration at mainly inner leaves (the symptom of the tipburn) is often observed in the lettuce plants of the tipburn-susceptible line. The discoloration is small in the lettuce plant having medium tipburn-resistance. The discoloration is very small or not observed at all in the lettuce plant line having high tipburn-resistance.

The lettuce plant has 1 to 18 chromosomes (18 chromosomes are 9 pairs of chromosomes). Each chromosome in the lettuce plant may be determined, for example, by comparing the base sequence information of the genome of the subject lettuce plant with that of Salinas based on that of the genus Lactuca sativa (the name of the breed: Salinas). The comparison aforementioned may be carried out, for example, by using the analysis software such as BLAST and FASTA. The base sequence information of the genome of Salinas can be available from the lettuce genome data base described below.
(1) Lettuce genome data base: Lettuce Last_Salinas_v7(GCF_002870075.2_Last_Salinas_v7_genomic.chr1-9_unplaced.fna)
(2) The acquisition source WEB site of the lettuce genome data base: https://www.ncbi.nim.nih.gov/assembly/GCA_002870075.2 #/def

The resistance marker of the present invention includes the resistance gene locus on the 4th chromosome, but the lettuce plant having the resistance gene locus may have the resistance gene locus on the 4th chromosome, for example, on any chromosome except the 4th chromosome instead of the 4th chromosome. Namely, the lettuce plant having the resistance gene locus may have the resistance gene locus on the 4th chromosome on any of the 1st chromosome, the 2nd chromosome, the 3rd chromosome, the 5th chromosome, the 6th chromosome, the 7th chromosome, the 8th chromosome, and the 9th chromosome.

Any of the resistance markers of the present invention may be heterozygous or homozygous, for example, at the resistance gene locus on the 4th chromosome. In the case of the latter, the resistant lettuce plant may include at least one resistance marker on the chromosome except the 4th chromosome and, for example, one resistance gene locus on the chromosome except the 4th chromosome or two resistance gene loci on the chromosome except the 4th chromosome. When the resistant lettuce plant includes two resistance gene loci on the chromosome except the 4th chromosome, the resistant lettuce plant may include, for example, two resistance gene loci aforementioned on the same chromosome or on the different chromosomes.

The tipburn-resistance gene locus means the Quantitative Traits Loci or the gene region imparting tipburn-resistance. The Quantitative Traits Loci (QTL) generally means the chromosome region involved in the expression of the quantitative traits. The QTL can be specified by using the molecular marker indicating the specific locus on the chromosome. The technique specifying the QTL by using the molecular marker is well known in the art.

In the present invention, the molecular marker used for specifying the resistance gene locus is not particularly limited. The molecular markers include SNP marker, AFLP (amplified fragment length polymorphism) marker, RFLP (restriction fragment length polymorphism), microsatellite marker, SCAR (sequence-characterized amplified region) marker, KASP (Kompetitive Allele Specific PCR) marker, and CAPS (cleaved amplified polymorphic sequence) marker. In this specification, the resistance gene locus is specified by the SNP marker for convenience. For example, one SNP may be used as the SNP marker or the combination of two or more SNP may be used as the SNP marker.

### (1) Identification by SNP Marker

The lettuce plant having tipburn resistance of the present invention has at least one polynucleotide of the following (a), (b), (c), and (d) on the gene:
(a) polynucleotide including polymorphism that the 271576390th cytosine base from the first of the 4th chromosome is thymine base and wherein bases of 50 bp before and after the thymine base make up a base sequence having no less than 80% identity to that of sequence number 1;
(b) polynucleotide including polymorphism that the 274188377th cytosine base from the first of the 4th chromosome is thymine base and wherein bases of 50 bp before and after the thymine base make up a base sequence having no less than 80% identity to that of sequence number 2;
(c) polynucleotide including polymorphism that the 274188380th thymine base from the first of the 4th chromosome is guanine base and wherein bases of 50 bp before and after the guanine base make up a base sequence having no less than 80% identity to that of sequence number 3; and
(d) polynucleotide including polymorphism that the 274188382th adenine base from the first of the 4th chromosome is cytosine base and wherein bases of 50 bp before and after the cytosine base make up a base sequence having no less than 80% identity to that of sequence number 4.

The SNP markers with which the polynucleotide of the (a), (b), (c), and (d) described above are specified are referred to as SNP (a), SNP (b), SNP (c), and SNP (d), respectively, or sometimes as NC_056626.1-271576390, NC_056626.1-274188377, NC_056626.1-274188380, and NC_056626.1-274188382, respectively.

“NC_056626.1” means the 4th chromosome and the number following the hyphen, for example, “271576390” means the 271576390th from the first of the 4th chromosome.

Note that for the SNP analysis, it is possible to refer to, for example, the following Reference Document 1. NC_056626.1-271576390, NC_056626.1-274188377, NC_056626.1-274188380, and NC_056626.1-274188382 are the SNP markers which the present inventors newly identify. The skilled persons in the art can specify the position on which SNP marker is mounted based on the base sequence including the SNP markers described below.
Reference Document 1: Kozik A et al., “The alternative reality of plant mitochondrial DNA: One ring does not rule them all.”, PLoS Genet, 2019 August; 15(8):e1008373
Reference Document 2: Reyes-Chin-Wo S et al., “Genome assembly with in vitro proximity ligation data and whole-genome triplication in lettuce.”, Nat Commun, 2017 Apr. 12; 8:14953
Reference Documents 1 and 2 are described at “Publications” of “https://www.ncbi.nlm.nih.gov/bioproject/PRJNA173551/”.

The NC_056626.1-271576390 (hereinafter also referred to as “SNP (a)”) means the polymorphism wherein the 271576390th base from the first of the 4th chromosome is T. Namely, this means that when the 271576390th base from the first of the 4th chromosome is T, the lettuce plant is tipburn-resistant and that when the 271576390th base from the first of the 4th chromosome is the base except T (for example, C), the lettuce plant is tipburn-susceptible. The base sequence described above can be obtained from the lettuce plant deposited under FERM AP-22442 described below. The SNP (a) can be also specified from the known information on the data base of, for example, the website described above and the like.

For your information, the sequence information of the SNP (a) (sequence number 1) is shown. The polymorphism wherein the base enclosed in parentheses is T is shown. The following sequence information is the sequence information obtained by cutting out the 50 bps bases before and after the base enclosed in parentheses.

5′_ATAAAACCATAAGCAGCTGCAGCATAAACAAGTGCTTGTTCATCTG
GAGA[T]TCCCCTTGATAATCAATCAACTTCTCATTGGGATCAGATGTG
TCAACAAC_3′

The NC_056626.1-274188377 (hereinafter also referred to as “SNP (b)”) means the polymorphism wherein the 274188377th base from the first of the 4th chromosome is T. Namely, this means that when the 274188377th base from the first of the 4th chromosome is T, the lettuce plant is tipburn-resistant and that when the 274188377th base from the first of the 4th chromosome is the base except T (for example, C), the lettuce plant is tipburn-susceptible. The base sequence of the sequence number 2 can be obtained from, for example, the lettuce plant deposited under FERM AP-22442. The SNP (b) can be also specified from the known information on the data base of, for example, the website described above and the like.

For your information, the sequence information of the SNP (b) (sequence number 2) is shown. The polymorphism wherein the base enclosed in parentheses is T is shown. The following sequence information is the sequence information obtained by cutting out the 50 bps bases before and after the base enclosed in parentheses.

5′_GTCTTTTGTTCAAAGCCCAAATCTGTTGTTCCCTTGGATATATACT
AAAA[T]TCTGACCATTTTCTTCTGCTTTCACAACATGCGAAAAGACCT
CCGGGGGT_3′

The NC_056626.1-274188380 (hereinafter also referred to as “SNP (c)”) means the polymorphism wherein the 274188380th base from the first of the 4th chromosome is G. Namely, this means that when the 274188380th base from the first of the 4th chromosome is G, the lettuce plant is tipburn-resistant and that when the 274188380th base from the first of the 4th chromosome is the base except G (for example, T), the lettuce plant is tipburn-susceptible. The base sequence described above can be obtained from the lettuce plant deposited under FERM AP-22442. The SNP (c) can be also specified from the known information on the data base of, for example, the website described above and the like.

For your information, the sequence information of the SNP (c) (sequence number 3) is shown. The polymorphism wherein the base enclosed in parentheses is G is shown. The following sequence information is the sequence information obtained by cutting out the 50 bps bases before and after the base enclosed in parentheses.

5′_TTTTGTTCAAAGCCCAAATCTGTTGTTCCCTTGGATATATACTAAA
ACTC[G]GACCATTTTCTTCTGCTTTCACAACATGCGAAAAGACCTCCG
GGGGTAGT_3′

The NC_056626.1-274188382 (hereinafter also referred to as “SNP (d)”) means the polymorphism wherein the 274188382th base from 5′ terminal of the 4th chromosome is C. Namely, this means that when the 274188382th base from the first of the 4th chromosome is C, the lettuce plant is tipburn-resistant and that when the 274188382th base from the first of the 4th chromosome is the base except C (for example, A), the lettuce plant is tipburn-susceptible. The base sequence described above can be obtained from the lettuce plant deposited under FERM AP-22442. The SNP (d) can be also specified from the known information on the data base of, for example, the website described above and the like.

For your information, the sequence information of the SNP (d) (sequence number 4) is shown. The polymorphism wherein the base enclosed in parentheses is C is shown. The following sequence information is the sequence information obtained by cutting out the 50 bps bases before and after the base enclosed in parentheses.

5′_TTGTTCAAAGCCCAAATCTGTTGTTCCCTTGGATATATACTAAAAC
TCTG[C]CCATTTTCTTCTGCTTTCACAACATGCGAAAAGACCTCCGGG
GGTAGTGG_3′

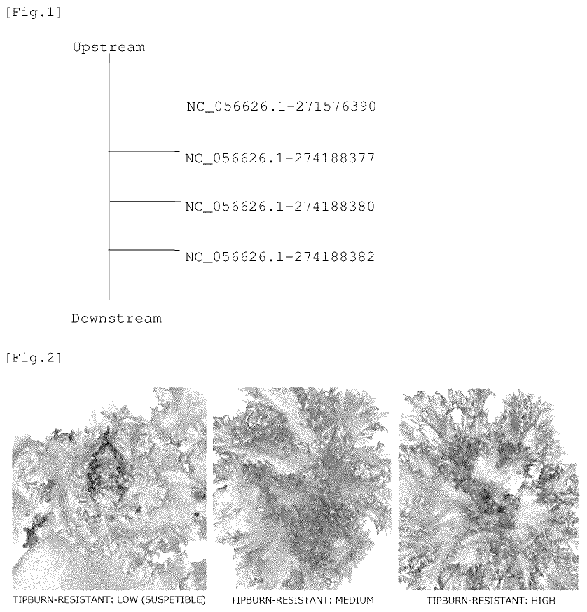

As shown in FIG. 1, the SNP markers of the present invention, NC_056626.1-271576390, NC_056626.1-274188377, NC_056626.1-274188380, and NC_056626.1-274188382 are mounted from the upstream side (NC_056626.1-271576390 side) to the downstream side (NC_056626.1-274188382 side) in this order, for example, on the 4th chromosome of the lettuce plant.

It can be said that the lettuce plant which is the polymorphism of the lettuce plant deposited under FERM AP-22442 is tipburn-resistant.

The number of the SNP markers in the resistance gene locus is not particularly limited. For example, the resistance gene locus may have anyone, two, three, or four of the SNP markers. Note that the relevance of these 4 kinds of polymorphisms (SNP markers) to the tipburn resistance have not been reported so far and that these polymorphisms are the novel polymorphisms relevant to the tipburn resistance found by the present inventors for the first time.

The SNP markers can be used alone or in combination and the combination is not particularly limited when used in combination. For example, The SNP markers can be used alone or in combination as described below.
SNP (a)
SNP (b)
SNP (c)
SNP (d)
The combination of SNP (a) and SNP (b)
The combination of SNP (a) and SNP (c)
The combination of SNP (a) and SNP (d)
The combination of SNP (b) and SNP (c)
The combination of SNP (b) and SNP (d)
The combination of SNP (c) and SNP (d)
The combination of SNP (a), SNP (b) and SNP (c)
The combination of SNP (a), SNP (b) and SNP (d)
The combination of SNP (a), SNP (c) and SNP (d)
The combination of SNP (b), SNP (c) and SNP (d)
The combination of SNP (a), SNP (b), SNP (c) and SNP (d)

Among the above combinations, “The combination of SNP (a), SNP (b), SNP (c) and SNP (d)” is the preferable SNP marker because the correlativeness of the combination with the tipburn resistance is higher.

The tipburn-resistant lettuce plant does not have to have the same base sequence as that of the sequence number 1, 2, 3, and 4. The identity to the base sequence of the sequence number 1, 2, 3, and 4 may be, for example, no less than 80%, 85%, 90%, 95%, and 99%.

The 271576390th base(T) from the first of the 4th chromosome is the base corresponding to the polymorphism of the SNP (a). The 274188377th (T) from the first of the 4th chromosome is the base corresponding to the polymorphism of the SNP (b). The 274188380th base (G) from the first of the 4th chromosome is the base corresponding to the polymorphism of the SNP (c). The 274188382th base (C) from the first of the 4th chromosome is the base corresponding to the polymorphism of the SNP (d).

### (2) Polynucleotide Having Base Sequence Including SNP Marker

The resistance gene locus may be specified by the base sequence including the SNP marker. The resistance gene locus, for example, may consist of or include the base sequence aforementioned.

The base sequence may be the base sequence including at least one of the SNP (a), the SNP (b), the SNP (c), and the SNP (d). The base sequence includes the base sequences of the sequence number 1, 2, 3, and 4 described above but may be the base sequence including any one of the base sequence of the sequence numbers 1, 2, 3, and 4.

The prescribed base sequence including the SNP marker can be obtained from the lettuce plant deposited under FERM AP-22442. The base sequence including the SNP marker do not have to be identical to that of the lettuce plant deposited about all bases. As long as the SNP (a), the SNP (b), the SNP (c), and the SNP (d) can be specified, the other bases (namely, the bases except the marker) may be different from that of the lettuce plant deposited.

Namely the polynucleotide may be the polynucleotide consisting of the base sequence where one or a part of the bases are deleted, substituted, inserted, and added or the polynucleotide consisting of the base sequence having no less than 80% identity to that of each polynucleotide and wherein the polymorphism or the specific base described above of each polynucleotide is conserved.

Note that the identity between two sequences can be identified by the alignment (the same shall apply hereinafter). Specifically, the identity can be calculated, for example, based on the default parameter using the analysis software such as BLAST.

### (3) Base Sequence of Region Between Sites of Two SNP Markers

The resistance gene locus can be specified by the base sequence of the region between the sites of two SNP markers among four SNP markers.

The base sequence of the region between the sites of two SNP markers is not particularly limited and include, for example, the base sequence of the region between the sites of two SNP markers selected from the group consisting of NC_056626.1-271576390, NC_056626.1-274188377, NC_056626.1-274188380, NC_056626.1-274188382.

For the base sequence of the region between the sites of two SNP markers, for example, the base sequence of the region between the sites of two SNP markers corresponding in the lettuce plant deposited under FERM AP-22442 (hereinafter referred to as “deposited line”) described below can be referred to. When the base sequence of the deposited line is referred to, the base sequence of the region between the sites of two SNP markers agree with, for example, the base sequence of the deposited line perfectly or partly. In the latter case, the lettuce plant having the resistance gene locus specified by the base sequence of the region between the sites of two SNP markers only have to show the tipburn resistance. When the resistance gene locus is specified by the base sequence of the region between the sites of two SNP markers, it also can be said that the resistance gene locus is mounted on, for example, the region between the sites of two SNP markers.

As described above the upstream end and the downstream end of the region can be specified, for example, by the sites of two SNP markers. The region may be between the sites of two SNP markers and, for example, may include both or one of the sites of two SNP markers or may not. When the region includes the site of the SNP marker, the upstream end and the downstream end of the region are the sites of the SNP markers but the bases of the upstream end and the downstream end, for example, may be the base enclosed in parentheses described above or the base except the base enclosed in parentheses.

Two SNP markers by which the region is defined are not particularly limited and, for example, include the following combination.
The combination of the SNP (a) and (b)
The combination of the SNP (a) and (c)
The combination of the SNP (a) and (d)
The combination of the SNP (b) and (c)
The combination of the SNP (b) and (d)
The combination of the SNP (c) and (d)

When the resistance gene locus is specified by the base sequence of the region between the sites of two SNP markers, the resistance gene locus preferably has the SNP marker mounted on the region in the base sequence of the region. Specifically, the resistance gene locus preferably has at least one SNP marker selected from the group consisting of NC_056626.1-274188377 and NC_056626.1-274188380 in the base sequence of the region.

The SNP marker mounted on the region may be, for example, one or both of the sites of two SNP markers by which the region is specified on the chromosome or may be the SNP marker mounted between the sites of two SNP markers by which the region is specified. The former is also referred to as the terminal SNP marker of the region and the latter is also referred to as the internal SNP marker of the region. The SNP marker mounted on the region may be, for example, both of the terminal SNP marker and the internal SNP marker of the region.

The internal SNP markers of the region include, for example, the SNP marker mounted between the site of the SNP marker of the upstream side and the site of the SNP marker of the downstream side by which the region is defined and can be determined, for example, based on the position shown in FIG. 1 on which the SNP marker is mounted according to circumstance. The number of the SNP markers between the sites of two SNP markers may be, for example, no less than one. The specific examples include all the SNP markers mounted between the sites of the SNP markers by which the region is defined.

The combinations of the base sequence of the region between the sites of two SNP markers and the SNP markers in the base sequence of the region are not particularly limited and, for example, include the combination of the following conditions (i), (ii), and (iii).

<The Condition (i)>

The base sequence of the region between the sites of the SNP (a) and the SNP (c) is included and there is the SNP marker of the SNP (b) in the base sequence of the region on the chromosome.

<The Condition (ii)>

The base sequence of the region between the sites of the SNP (b) and the SNP (d) is included and there is the SNP marker of the SNP (c) in the base sequence of the region on the chromosome.

<The Condition (iii)>

The base sequence of the region between the sites of the SNP (a) and the SNP (d) is included and there are the SNP markers of the SNP (b) and the SNP (c) in the base sequence of the region on the chromosome.

The resistance gene locus is, for example, the tipburn-resistance gene locus on the 4th chromosome of the lettuce plant deposited under FERM AP-22442 described below.

### 2. Lettuce Plant Having Tipburn-Resistance

By using the tipburn-resistance marker of the present invention, for example, the tipburn-resistance can be given to the lettuce plant. In the present invention the degree of the tipburn-resistance can be indicated by the level of the resistance calculated from the resistance index of the lettuce plant by the method in Example 1 described below. For the calculation of the resistance evaluation index by the method the description in Example 1 described below can be employed. For example, the resistance evaluation index of less than 5 can be set to be susceptible and the resistance evaluation index of no less than 5 can be set to be resistant. When the tipburn-resistance is determined by using the resistance evaluation index, the resistance evaluation index used may be the resistance evaluation index of 1 lettuce plant or the mean of the resistance evaluation indexes of no less than two lettuce plants. The latter is preferable. In the case of the latter, the number of the lettuce plants used for determining the tipburn-resistance is not particularly limited and is, for example, the number testable by the statistical test for comparing with the tipburn-susceptible lettuce plant. The specific examples include 5 to 20 plants.

The tipburn-resistant lettuce plant of the present invention is characterized by having the tipburn-resistance gene locus on the 4th chromosome as described above. The tipburn-resistant lettuce plant of the present invention is characterized by having the tipburn-resistance gene locus on the 4th chromosome and other composition and condition are not particularly limited. The tipburn-resistant lettuce plant of the present invention has the tipburn-resistance marker of the present invention including the tipburn-resistance gene locus and thus, for example, the description of the tipburn-resistance marker for the lettuce plant of the present invention can be employed for the tipburn-resistant lettuce plant. In the present invention the tipburn-resistance gene locus on the 4th chromosome can be, for example, replaced with the tipburn-resistance marker of the present invention.

The tipburn-resistant lettuce plant of the present invention shows resistance against the tipburn.

As described above, in the tipburn-resistant lettuce plant of the present invention, the tipburn-resistance is given by the tipburn-resistance gene locus on the 4th chromosome. The tipburn-resistant lettuce plant of the present invention has the resistance gene locus on the 4th chromosome but, for example, may have the tipburn-resistance gene locus on the 4th chromosome on any one of the chromosomes except the 4th chromosome. Namely the lettuce plant having the resistance gene locus may have the resistance gene locus on the 4th chromosome on any one of the 1st chromosome, the 2nd chromosome, the 4th chromosome, the 5th chromosome, the 6th chromosome, the 7th chromosome, the 8th chromosome, the 9th chromosome, the 10th chromosome, the 11th chromosome, the 12th chromosome, the 13th chromosome, the 14th chromosome. The 15th chromosome, the 16th chromosome, the 17th chromosome, and the 18th chromosome.

The tipburn-resistant lettuce plant of the present invention may have, for example, one resistance gene locus or two or more resistance gene loci. As a concrete example, regarding to a pair of chromosomes, one chromosome may include the resistance gene locus (heterozygous) or both of chromosomes may include the resistance gene loci (homozygous). The latter is preferable because the tipburn-resistance improve more.

For the resistance gene locus in the tipburn-resistant lettuce plant of the present invention, for example, the description of the tipburn-resistance gene locus in the tipburn-resistance marker for the lettuce plant of the present invention can be employed.

The tipburn-resistant lettuce plant of the present invention include the lettuce plant (Lactuca sativa) deposited under FERM AP-22442 or the progeny line thereof. The progeny line, for example, have the resistance gene locus. The information of the deposit is as follow.
Kind of deposit: National deposit
Name of depositary institution: National Institute of Technology and Evaluation, International Patent Organism Depositary
Address:FERM AP-22442 #120, 2-5-8, Kazusakamatari, Kisarazu-shi, Chiba, 292-0818, Japan
Sign for identifying: YLA-1
Date of receipt: Feb. 1, 2022

The tipburn-resistant lettuce plant of the present invention can be produced, for example, by introducing the resistance gene locus to the lettuce plant. The method for introducing the resistance gene locus to the lettuce plant is not particularly limited and, for example, include the crossing with the resistant lettuce, the embryo culture or the conventionally known genetically engineering method. The resistance gene locus to be introduced include the resistance gene locus described above. When introducing by the crossing with the resistant lettuce plant, the resistant lettuce plant preferably includes, for example, the resistance gene loci homozygously.

Regarding to the tipburn-resistant lettuce plant of the present invention the characteristics except the tipburn-resistance, for example, the trait, the ecological characteristics and the like are not particularly limited.

The tipburn-resistant lettuce plant of the present invention may have other resistance in addition.

In the present invention, “plant body” may mean either of the plant individual which indicate whole plant or the part of the plant individual. The parts of the plant individual include the organ, the tissue, the cell, or the vegetative propagated body and may be any one of them. The organs include the petal, the corolla, the flower, the pollen, the leaf, the seed, the fruit, the stem, the root. The tissue is, for example, the part of the organ. The part of the plant body may be, for example, one kind of organ, tissue, and/or cell or two or more kinds of organs, tissue, and/or cells.

### 3. Production Method for Tipburn-Resistant Lettuce Plant

Next, the production method for the tipburn-resistant lettuce plant of the present invention is described. Note that the following methods are examples and the present invention is not limited to these methods. In the present invention the production method can be said to be, for example, the raising method. In the present invention the tipburn-resistance gene locus can be put into the tipburn-resistance marker and the description can be employed.

As described above, the production method for the tipburn-resistant lettuce plant of the present invention is characterized by including the following steps (A) and (B).
(A) The step of crossing the tipburn-resistant lettuce plant of the present invention with other lettuce plant.
(B) The step of picking up the tipburn-resistant lettuce plants from the lettuce plants obtained by the step (A) or the progeny line thereof.

The production method of the present invention is characterized by using the tipburn-resistant lettuce plant of the present invention as the parents. Other steps and conditions are not limited at all. For the production method of the present invention, for example, the descriptions of the tipburn-resistance marker, the tipburn-resistant lettuce plant and the like of the present invention can be employed.

In the step (A) the tipburn-resistant lettuce plant used as the first parent only need to be the tipburn-resistant lettuce plant of the present invention. The tipburn-resistant lettuce plant is preferably, for example, the lettuce plant deposited under FERM AM-22442 described above or the progeny line thereof. In the step (A) the tipburn-resistant lettuce plant used as the first parent can be obtained, for example, by the screening method of the present invention described below. Thus, the tipburn-resistance lettuce plant may be prepared, for example, by the following step (C) of picking up from the lettuce plant being tested (also referred to as “candidate lettuce plant”) before the step (A).

The step (C) is as follow.

(C) The step of picking up the tipburn-resistant lettuce plant of the present invention from the lettuce plant being tested.

In the step (C) the picking up of the tipburn-resistant lettuce plant can be said to be the picking up of the lettuce plant having the tipburn-resistance gene locus. Thus the step (C) can be carried out by the following step (C1) and the step (C2).

(C1) The step of detecting the presence/absence of the tipburn-resistance gene locus on the chromosome of the lettuce plant being tested.

(C2) The step of picking up the lettuce being tested based on the presence of the tipburn-resistance gene locus as the tipburn-resistant lettuce plant.

As described above, the picking up in the step (C) is, for example, the picking up of the lettuce plant having the tipburn-resistance gene locus. Specifically, the tipburn-resistant lettuce plant can be picked up by detecting the tipburn-resistance gene locus in the lettuce being tested. In the step (C2), for example, when the resistance gene locus is mounted on the one of a pair of the chromosomes, the lettuce plant being tested may be picked up as the tipburn-resistant lettuce plant. When the resistance gene loci are mounted on the both of a pair of the chromosomes, the lettuce plant being tested may be picked up as the tipburn-resistant lettuce plant. The latter is preferable. The detection of the tipburn-resistance gene locus in the step (C1) can be carried out, for example, by using the SNP marker, the base sequence including the SNP marker, the base sequence of the region between the sites of two SNP markers, and the combination thereof.

Regarding to the picking up in the step (C) the following concrete examples are given, but the picking up is not limited to the examples. For the tipburn-resistance gene locus the description in the tipburn resistance marker of the present invention can be employed.

### (1) Specification by SNP Marker

The picking up in the step (C) is the picking up of the lettuce plant having the tipburn-resistance gene locus specified by at least one SNP marker selected from the group consisting of the SNP (a), the SNP (b), the SNP (c), and the SNP (d). The SNP marker to be selected is not particularly limited and, for example, for it the description of “(1) Specification by SNP marker” in the tipburn-resistance marker of the present invention can be employed.

### (2) Specification by Base Sequence Including SNP Marker

The picking up in the step (C) is the picking up of the lettuce plant having the tipburn-resistance gene locus specified by polynucleotide including at least one of the SNP (a), the SNP (b), the SNP (c), and the SNP (d). For polynucleotide including at least one of the SNP (a), the SNP (b), the SNP (c), and the SNP (d), for example, the description of “(2) Specification by the base sequence including SNP marker” in the tipburn-resistance marker of the present invention can be employed.

(3) Specification by Base Sequence of Region between Sites of Two SNP Markers

The picking up in the step (C) is the picking up of the lettuce plant having the tipburn-resistance gene locus including the base sequence of the region between the sites of two SNP markers selected from the group consisting of the SNP (a), the SNP (b), the SNP (c), and the SNP (d). For the base sequence of the region between the sites of two SNP markers, for example, the description of “(3) Specification by base sequence of region between sites of two SNP markers” in the tipburn-resistance marker of the present invention can be employed.

The picking up in the step (C) may be the picking up of the lettuce plant having the tipburn-resistance gene locus satisfying at least one of the conditions (i), (ii), (iii), and (iv) described above.

The chromosome on which the presence/absence of the tipburn-resistance gene locus is detected is preferably the 4th chromosome.

In the step (A) the lettuce plant used as the other parent is not particularly limited and may be, for example, the known lettuce plant having or not having tipburn resistance, the lettuce plant having or not having other resistance or the tipburn-resistant lettuce plant of the present invention.

In the step (A) the crossing method of the tipburn-resistant lettuce plant with other lettuce plant is not particularly limited and the well-known method can be adopted.

In the step (B) the subject from which the tipburn resistant lettuce plant is picked up may be, for example, the lettuce plant obtained in the step (A) or further the progeny line obtained from the lettuce plant. Specifically, the subject may be, for example, the F1 lettuce plant obtained by the crossing of the step (A) or the progeny line thereof. The progeny line may be, for example, the progeny line by the inbreeding or the backcross of the F1 lettuce plant obtained by the crossing of the step (A), or the lettuce plant obtained by the crossing of the F1 lettuce plant with other lettuce plant.

In the step (B) the picking up of the tipburn-resistant lettuce plant can be carried out, for example, by confirming the tipburn-resistance directly or indirectly.

In the step (B) the direct confirmation can be carried out by evaluating the tipburn-resistance for the F1 lettuce plant obtained or the progeny line thereof with the resistance evaluation index. Specifically, for example, for the F1 lettuce plant or the progeny line thereof, for example, the tipburn-resistance can be confirmed by evaluating with the resistance evaluation index. In this case, for example, the F1 lettuce plant or the progeny line thereof whose the resistance evaluation index is no less than 5 can be picked up as the tipburn-resistant lettuce plant.

In the step (B) the picking up by the indirect confirmation is carried out by the following step (B1) and the step (B2).

(B1) The step of detecting the presence/absence of the tipburn-resistance gene locus on the chromosome for the lettuce plant obtained by the step (A) or the progeny line thereof.

(B2) The step of picking up the lettuce plant obtained by the step (A) or the progeny line thereof as the tipburn-resistant lettuce plant based on the presence of the tipburn-resistance gene locus.

The picking up of the tipburn-resistant lettuce plant by the indirect confirmation in the step (B) is similar to, for example, the method described in the step (C) and is carried out by the detection of the presence/absence of the tipburn-resistance gene locus, more specifically, by the detection of the presence/absence of the tipburn-resistance gene locus by using the tipburn-resistance marker.

The production method of the present invention preferably includes the raising of the tipburn-resistant lettuce plant picked up in the step (B).

As described above, the lettuce plant confirmed to have tipburn-resistance or the progeny line thereof can be picked up as the tipburn-resistant plant.

The production method of the present invention may include the seed production step of gathering the seeds from the progeny line obtained by the crossing further.

The production method of the present invention may include, for example, only the step (A).

### 4. Screening Method of Tipburn-Resistant Lettuce Plant

The screening method of the tipburn-resistant lettuce plant of the present invention (hereinafter referred to as “the screening method”) is characterized by including the step of picking up the lettuce plant including the tipburn-resistance gene locus on the 4th chromosome as the tipburn-resistance marker for the lettuce plant from the lettuce plant being tested as the parent to produce the tipburn-resistant lettuce plant by the crossing.

The screening method of the present invention is characterized by including the step of picking up the lettuce plant including the tipburn-resistance gene locus on the 4th chromosome as the tipburn-resistance marker for the lettuce plant from the lettuce plant being tested and other steps and conditions are not limited. By using the screening method of the present invention, the tipburn-resistant parent can be obtained with the tipburn-resistance marker of the present invention. For the screening method of the present invention, for example, the description of the tipburn-resistance marker, the tipburn-resistant lettuce plant, the production method and the like can be employed.

For the picking up of the parent, for example, the description of the step (C) in the production method of the tipburn-resistant lettuce plant of the present invention can be employed.

### 5. Method for Giving Lettuce Plant Tipburn-Resistance

The method for giving the lettuce plant the tipburn-resistance of the present invention is characterized by including the step of introducing the tipburn-resistance gene locus on the 4th chromosome into the lettuce plant. The method for giving of the present invention is characterized by including the step of introducing the tipburn-resistance gene locus on the 4th chromosome into the lettuce plant and other steps and conditions are not particularly limited. According to the method for giving of the present invention by introducing the tipburn-resistance gene locus on the 4th chromosome, namely the tipburn-resistance marker of the present invention the tipburn-resistance can be given to the lettuce plant. For the method for giving, for example, the description of the tipburn-resistance marker, the tipburn-resistant lettuce plant, the production method, the screening method and the like of the present invention can be employed.

In the introduction step the method for introducing the tipburn-resistance gene locus on the 4th chromosome is not particularly limited. The introduction methods include the crossing with the resistant lettuce plant, the embryo culture or the well-known genetically engineering method. The tipburn-resistance gene locus to be introduced can include the tipburn-resistance gene locus described above. When introducing by the crossing with the tipburn-resistant lettuce plant, the tipburn-resistant lettuce plant preferably includes the tipburn-resistance gene loci homozygously.

### EXAMPLES

Hereinafter, the present invention is described in detail with reference to examples but is not limited to the embodiments described in Examples.

### Example 1

It was confirmed that the novel tipburn-resistant lettuce plant showed the tipburn-resistance. The analysis of the mode of inheritance of the tipburn-resistance gene locus and the specification of the novel tipburn-resistance gene locus were carried out.

To develop the novel lettuce plant exhibiting the tipburn-resistance by using the seeds of many kinds of the lettuce lines raised successively to gather at the plant factory in Center for Environment, Health and Field Science Chiba University used by Leaf Labo Co., Ltd. and YOSHINOYA HOLDINGS CO., LTD. for the joint studies, the breeding was carried out and the tipburn-resistance was tested. As the result, the novel tipburn-resistant lettuce line (Lactuca sativa) which show tipburn-resistance was obtained. Hereinafter the tipburn-resistant lettuce plant was referred to as the parent line.

Separately 118 lines of Recombinant inbred lines (hereinafter also referred to as RILs) of F5 generations obtained by crossing the tipburn-resistant line with the tipburn-susceptible line were produced. The markers relevant to the tipburn-resistance gene were searched as described below at Kazusa DNA Research Institute.

### (1) DNA Extraction and Quality Confirmation

The DNA extraction was carried out by using the automatic DNA extraction apparatus oKtopure™ (LGC Biosearch Technologies).

The QC of DNA was carried out by the concentration measurement. The concentration of all samples was measured by the spectrophotometer (NanoDrop™ 8000 Thermo Fisher) and the concentration of a part of the samples was measured by the fluorophotometer (Qibit BR: Thermo Fisher Scientific).

(2) Preparation of ddRAD-Seq Library

The analysis of ddRAD-Seq was carried out according to the method of Shirasawa et al. (2016). After cleaving all of the DNA samples with two kinds of restriction enzyme (Pst1 and Msp1), the adapter was bound to the cleaved terminal and then the purification was carried out by using the magnetic beads to remove the short chain DNA unreacted. PCR was carried out by using the purified DNA as the mold and the primer having the tag sequence to identify each individual. The amplified fragments of each sample obtained were mixed and the DNA of 300 to 900 bp were fractionated and collected by the DNA Size Selection System Blue Pippin (Sage Science).

### (3) Acquisition of Sequence Data

The sequence analysis was carried out by next generation sequencer (illumina). The reagent NextSeq500 HighOutpit Kit v2.5 was used and the paired end reads of 75 bp were obtained.

### (4) Information Analysis

FASTQC (Andrews 2010), PRINSEQ (Schmieder and Edwards 2011), and fastx_clipper (http://hannonlab.csh1.edu/fastx_toolkit) were used for the quality examination and the trimming of the sequence. Bowtie2 program (Langmead and Salzberg 2012) was used for the mapping of the sequence. The bcftools mpilneup/call command (Li et al. 2009) of SAMtools program was used for the variant calls. The lettuce Last_Salinas_v7 GCF_002870075.2_Last_Salinas_v7_genomic.chr1-9_unplaced.fna “https://www.ncbi.nlm.nih.gov/assembly/GCA_002870075.2 #/def” was used as the reference sequence used for the mapping. VCFtools program (Danecek et al. 2011) was used for the variant filtering. The filtering condition was set as follow.
The filtering condition 1:
QUAL 999
DP no less than 5 and no more than 200
GQ no less than 10 Multi allele were removed
The base of Ref was not N.
The filtering condition 2:
max-missing (mm) 0.3, 0.4, 0.5, 0.6, 0.7 (The proportion of the individual called in the all individuals was no less than the set value for each variant.)

The GWAS analysis was carried out by using the software TASSEL ver5.0 (https://www.maizegenetics.net/tassel) according to the general liner model (GLM) method.

Note that because the IDs which begin with NC or NW were used as the sequence name in the genome sequence file used for the GWAS analysis, the GWAS analysis and the detection of the variant (the tipburn-resistance marker) were carried out by using the IDs.
Examples: NC_056626.1 Lactuca sativa cultivar Salinas chromosome4, Last_Salinas_v7, whole genome shotgun sequence

These results were shown in Table 2. The number of the detected candidate significant tipburn-resistance marker was 44 when the p-value representing the risk rate was no more than 0.001. Then the Bonferroni correction was carried out and 4 candidate tipburn-resistance markers were detected on the 4th.chromosome when the p-value was less than 0.05.

TABLE 2

| | | | p < 0.05 |
|---|---|---|---|
| | Traits used in | | Bonferroni |
| Traits | GWAS analysis | p < 0.001 | correction |
| Tipburn-resistance | Tipburn- | 44 | 4 |
| evaluation index | resistance | | |

118 lines of Recombinant inbred lines (hereinafter also referred to as RILs) of F5 generations obtained by crossing the parent line with the fixed line of the tipburn-susceptible lettuce plant (also referred to as “the tipburn-susceptible line”) were produced.

The tipburn-resistance was evaluated for 118 lines. The method for evaluating the tipburn-resistance was as follow.

The evaluation of the tipburn-resistance was carried out at the plant factory with artificial light in Center for Environment, Health and Field Science Chiba University used by Leaf Labo Co., Ltd. and YOSHINOYA HOLDINGS CO., LTD. for the joint studies.

At the plant factory with artificial light 118 lines of Recombinant inbred lines of F5 generations were raised as described below. First the seeds were sowed in the urethane culture medium with 300 holes and the first raising seedling was performed for 15 days. Then the seedlings were transplanted to the planting panel with 26 holes and the second raising seedling was performed for 9 days. 24 days after sowing the seedlings were planted to be settled in the planting panel with 6 holes and raised for 14 days. The environmental condition was set as follow. The temperature: 25±1° C. during the light period and 21° C. during the dark period, The CO₂ concentration: 1,500 μmol·mol⁻¹ RH: 60±10% during the light period and 80% during the dark period. The culture solution was set to be Chiba University formulation for the lettuce plant, 1.45 dS·m⁻¹ of EC, and pH of 6.5. The white LED was used as the light source. The photosynthesis-effective photon flux density (PPFD) was set to be 300 μmol·m⁻² s⁻¹. The light period was set to be 20 hours. The tipburn-resistance evaluation was carried out from 1 day to 14 days after the settled plantation.

The tipburn-resistance evaluation was carried out according to the following criteria. The degree of the damage by the tipburn was shown in FIG. 2. The black-discolored parts mainly at inner leaves were observed in the tipburn-susceptible line and the line having medium tipburn-resistance in FIG. 2, which was tipburn. The degree of the damage by the tipburn can be evaluate to a certain degree visually as shown in FIG. 2. This is tipburn. The tipburn can be evaluate more accurately by carrying out the investigation including the growth rate and the number of the damaged leaves. For the evaluation of the tipburn-resistance, first 4 items of the date when the tipburn occurred (showing on what day after the settled plantation the tipburn occurred), the weight of the fresh plants per one plant when the tipburn occurred (g/plant), the rate of the tipburn-affected plant (%), and the ratio of the affected leaves (the number of the tipburn-affected leaves/the number of all leaves) were investigated and the evaluation points were given to the investigation results respectively. The average value of the evaluation points given to each of 4 investigation items was set to be the tipburn-resistance evaluation index. The table showing the correspondence between the evaluation criteria and the evaluation points of 4 investigation items and the tipburn-resistance evaluation index (the average value of the evaluation points) were shown in Table 3.

TABLE 3

| 4 evaluation criteria for the tipburn-resistance investigation | | | | | |
|---|---|---|---|---|---|
| | Weight of the fresh | | | | Tipburn- |
| Date when | plants per one plant | Rate of the | Ratio of the | | resistance |
| the tipburn | when the tipburn | tipburn- | affected | Evaluation | evaluation |
| occurred | occurred | affected plant | leaves | points | index |
| 1~2 | 10~ | 90~ | 50~ | 1 | 1 |
| 3 | 20~ | 80~ | 40~ | 2 | 2 |
| 5 | 30~ | 70~ | 30~ | 3 | 3 |
| 5 | 40~ | 60~ | 20~ | 4 | 4 |
| 6 | 50~ | 50~ | 10~ | 5 | 5 |
| 7 | 60~ | 40~ | 5~ | 6 | 6 |
| 8 | 70~ | 30~ | 1~ | 7 | 7 |
| 9~10 | 80~ | 20~ | 0.5~ | 8 | 8 |
| 11~12 | 90~ | 10~ | 0~ | 9 | 9 |
| None | None | 0 | 0 | 10 | 10 |

Regarding to 118 lines DNA was extracted. The polymorphism was analyzed by the GWAS analysis using NC_056626.1-274188382 the p-value representing the risk rate of which was lowest among 4 candidate markers.

These results were shown in the following Table 4. As shown in Table 4 when the genotype was the homozygous type (A) or the heterozygous type (H) of the NC_056626.1-274188382 of the resistance, the tipburn-resistance evaluation index was no less than 6. In contrast when the genotype was the homozygous type (B) of the susceptibility, the tipburn-resistance evaluation index was less than 4. From these results the tipburn-resistant lettuce plant of the present invention was proved to be effective against the tipburn. Additionally, it was revealed that NC_056626.1-274188382 could be used as the tipburn-resistance gene locus marker on the 4th chromosome.

TABLE 4

| Genotype | | | | | |
|---|---|---|---|---|---|
| [NC_056626.1- | Tipburn-resistance evaluation index | | | | |
| 274188382] | 1~1.9 | 2~3.9 | 4~5.9 | 6~7.9 | 8~10 |
| Homonzygous type (A) | 0 | 0 | 0 | 7 | 14 |
| of the resistance | | | | | |
| Heterozygous type (H) | 0 | 0 | 27 | 0 | 0 |
| Homonzygous type (B) | 27 | 43 | 0 | 0 | 0 |
| of the susceptibility | | | | | |

### Example 2

Regarding to the novel tipburn-resistant lettuce plant the novel tipburn-resistance gene locus was specified.

The backcross progeny BC1 line was obtained by crossing the parent line with the susceptible line. Note that regarding to each backcross progeny the lettuce plants including NC_056626.1-271576390 and NC_056626.1-274188382 heterozygously were picked up.

NC_056626.1-274188377 and NC_056626.1-274188380 which were the polymorphism near NC_056626.1-274188382 of Example 1 were newly designed as the SNP marker. Regarding to 118 lines and the BC1 line the bases of the polymorphism corresponding to NC_056626.1-271576390, NC_056626.1-274188377, NC_056626.1-274188380, and NC_056626.1-274188382 were specified.

Next, in 118 lines and the BC1 line 6 individuals (hereinafter referred to as “6 lines”) the genotype of the SNP marker of which was different from one another were picked up. Then 6 lines were inbred and the inbred progeny was obtained. Regarding to the inbred progeny the correspondence of the phenotype with the genotype was clarified in the same way as in Example 1. The result was shown in following Table 5. In Table 5 A represented the genotype having the SNP markers of the resistance homozygously, H represented the genotype having the SNP marker of the resistance heterozygously and B represented the genotype having the SNP markers of the susceptibility homozygously. As shown in following Table 5 in the inbred progeny of the individuals the genotype of which was homozygous type (A) or heterozygous type (H) of the NC_056626.1-274188377 of the resistance and the NC_056626.1-274188380 of the resistance, the individuals exhibiting the resistance were obtained. From these results it was confirmed that NC_056626.1-274188377 and NC_056626.1-274188380 were the SNP markers showing high correlativeness with the tipburn-resistance among the SNP markers. Because NC_056626.1-274188377 and NC_056626.1-274188380 showed high correlativeness, it was revealed that the region between the sites of NC_056626.1-274188382 and NC_056626.1-271576390 including the SNP markers showed high correlativeness with the tipburn-resistance.

In the parent lines of 118 lines, NC_056626.1-271576390, NC_056626.1-274188377, NC_056626.1-274188380, and NC_056626.1-274188382 specified as the SNP marker genotype of the lettuce plants exhibiting the tipburn-resistance (hereinafter referred to as the tipburn-resistant parent line) was confirmed to be the homozygous type (A) of the resistance like line 1 shown in Table 5 and the lettuce plants exhibiting the tipburn-resistance were deposited under FERM AP-22442. Hereinafter tipburn-resistant parent lines were also referred to as the deposited lines.

TABLE 5

| | SNP marker | | | | |
|---|---|---|---|---|---|
| | 271576390 | 274188377 | 274188380 | 274188382 | Phenotype of |
| | NC_056626.1 | NC_056626.1 | NC_056626.1 | NC_056626.1 | inbred lines |
| Line1 | A | A | A | A | Resistant |
| Line2 | B | A | A | A | Resistant |
| Line3 | B | H | H | H | Resistant/Susceptible |
| Line4 | A | H | H | H | Resistant/Susceptible |
| Line5 | B | B | B | B | Susceptible |
| Line6 | B | B | B | B | Susceptible |

Resistant: Only lettuce plants exhibiting resistance
Resistant/Susceptible: Mix of resistant and susceptible lettuce plants
Susceptible: Only lettuce plants exhibiting susceptible

### Example 3

From the results in Examples 1 and 2 it was revealed that the candidate tipburn-resistance marker obtained in Example 1 showed high correlation with the tipburn-resistance. Thus regarding to the tipburn-resistance SNP markers NC_056626.1-271576390, NC_056626.1-274188377, NC_056626.1-274188380, and NC_056626.1-274188382 the KASP analysis was carried out to pick up the KASP markers.

From the variants relevant to the tipburn-resistance gene obtained by using the tipburn-resistance SNP markers NC_056626.1-271576390, NC_056626.1-274188377, NC_056626.1-274188380, and NC_056626.1-274188382 obtained in Example 1 KASP markers were picked up and the confirmation test was carried out. The condition was as follow.

### Materials

136 DNA samples of the lettuce extracted in Example 1 and the variant information were used. The DNA samples were as follow. DNA sample: 118 individuals of F5 lines, 6 individuals of F1 6 individuals of each of the parent fixed lines

### Analysis and Detection

### (1) Information Analysis (Extraction of the Surrounding Sequence Information of the Candidate Region of SNPs) and Possibility Check of KASP Primer Design

The analysis was carried out regarding to the significant variants obtained in Example 1 (44 variants picked up with the p-value which was less than 0.001).
BLAST analysis: The sequences of 50 bp before and after the candidate SNPs were cut out and the BLAST analysis to the reference sequence was carried out.
Parameter: E-value≤1E−1

The number of the hit bases/the length of the sequences cut out before and after the SNP (101 bp): no less than 50%
Regarding to the said variants the list was prepared by arranging the sequence information according to the format for the possibility check of the primer design prescribed by LGC Biosearch Technologies.
The surrounding sequence information offered was sent to LGC Biosearch Technologies. The possibility of the primer design was examined and 3 SNPs were picked up.
The synthesis of the primers (KASP Assay mix) for the sequence including 3 SNPs picked up was entrusted.

### (2) Detection of SNPs

Reaction reagent kit: PACE^{T}M Genotyping Master Mix Standard ROX (3crbio) was used.
Detection device of fluorescence intensity: IntelliQube (LGC Biosearch Technologies) was used.
DNA typing: The automatic typing by the IntelliQube software and the visual modification were carried out.
Drawing: The genotype plot was made by the statistics software “R”.

### Analysis Results

### (1) Information Analysis Results (Extraction of Surrounding Sequence Information of Candidate Region of SNPs)

The sequences of 50 bp before and after 44 variants selected were cut out and the possibility of the KASP primer design was checked by LGC Biosearch Technologies. The duplication on the genome sequence was checked by the BLAST search.

Because regarding to all of 3 candidate variants first selected and shown in FIG. 6 the primer design was possible and the duplication was few, the primer synthesis for these variants was entrusted to LGC Biosearch Technologies.

TABLE 6

| NC_056626_1_ | NC_056626_1_ | NC_056626_1_ |
|---|---|---|
| 271576390 | 274188377 | 274188382 |

### (2) KASP Analysis

The primer synthesis for the sequence including 3 SNPs picked up was entrusted to LGC Biosearch Technologies and the KASP analysis was carried out. The allele discrimination ability of each marker was evaluated from the results of the parents and F1 (Table 7). It was revealed that the upstream SNC_056626_1_271576390 and the down stream SNC_056626_1_274188382 which were allele discriminable were the KASP markers usable for the actual breeding selection. From this KASP analysis the upstream SNC_056626_1_271576390 and the downstream SNC_056626_1_274188382 were developed as the practical markers. But any one of the markers may be used. Both or any one of NC_056626.1-274188377 and NC_056626.1-274188380 which were the markers between the practical markers may be used when the allele discrimination is possible.

The method or the kind of the analysis for the practical marker may be the other methods or kinds such as already well-known the AFLP analysis (marker) or the RFLP analysis (marker) and are not limited to the KASP analysis.

TABLE 7

| KASP marker | Allele discrimination ability |
|---|---|
| SNC_056626_1_271576390 | Allele discriminable |
| SNC_056626_1_274188377 | Allele unable to be discriminate |
| | [Alt side allele is not amplified.] |
| SNC_056626_1_274188382 | Allele discriminable |

The present invention was described with reference to the embodiment and Examples but is not limited to the embodiment and Example aforementioned. The various modifications of the constitution or the details of the present invention which the skilled parsons in the art can understand are possible within the scope of the present invention.

### INDUSTRIAL APPLICABILITY

As described above, by using the tipburn-resistance markers for the lettuce plant of the present invention, for example, the tipburn-resistant lettuce plant can be screened easily. The tipburn-resistant lettuce plant of the present invention, for example, can show the tipburn-resistance, for example, because of including the resistance gene locus. Thus, by using the tipburn-resistant lettuce plant of the present invention the decrease of the yield due to the tipburn can be avoid and the work labor for trimming the part damaged by tipburn can be largely decreased in the open-field cultivation under hot-dry environment, the plant factory where the growth rate is high and the tipburn is often observed and the like. Hence the present invention is very useful, for example, in the agricultural fields such as the breeding.

1. A tipburn-resistant lettuce plant comprising a tipburn-resistance gene locus on a 4th chromosome,
wherein the tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.

2. The tipburn-resistant lettuce plant according to claim 1, wherein the tipburn-resistant lettuce plant is a lettuce plant specified by FERM AP-22442 or a progeny line thereof.

3. The tipburn-resistant lettuce plant according to claim 1, wherein the tipburn-resistant lettuce plant is a plant body or a part thereof.

4. The tipburn-resistant lettuce plant according to claim 1, wherein the tipburn-resistant lettuce plant is a seed.

5. A method for producing a tipburn-resistant lettuce plant characterized by comprising following steps (A) and (B):
(A) a step of crossing the tipburn-resistant lettuce plant according to claim 1 with other lettuce plant,
(B) a step of picking up a tipburn-resistant lettuce plant from lettuce plants obtained in the step (A) or a progeny line thereof.

6. The method for producing a tipburn-resistant lettuce plant according to claim 5, comprising a following step (C) before the step (A):
(C) a step of picking up the tipburn-resistant lettuce plant from lettuce plants that have been tested.

7. The method for producing a tipburn-resistant lettuce plant according to claim 6, wherein the picking up in the step (C) is picking up of the tipburn-resistant lettuce plant including the tipburn-resistance gene locus on the 4th chromosome specified by at least one polynucleotide of followings (a), (b), (c), and (d),
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.

8. A method for giving lettuce plant tipburn-resistance comprising a step of introducing a tipburn-resistance gene locus on the 4th chromosome into a lettuce plant and that a tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.

9. The method for giving lettuce plant tipburn-resistance according to claim 8, wherein the tipburn-resistance gene locus is a tipburn-resistance gene locus specified by at least one of the polynucleotides of (a), (b), (c), and (d) of a lettuce plant specified by FERM AP-22442.

10. A method for screening a tipburn-resistant lettuce plant is comprising a step of picking up a lettuce plant including a tipburn-resistance gene locus on a 4th chromosome from lettuce plants that have been tested as parents for producing a tipburn-resistant lettuce plant by crossing and that the tipburn-resistance gene locus is specified by at least one polynucleotide of followings (a), (b), (c), and (d):
(a) polynucleotide including polymorphism that a 271576390th cytosine base from a first of the 4th chromosome is thymine base and wherein bases of 50 bp before and after the thymine base make up a base sequence having no less than 80% identity to a base sequence of sequence number 1;
(b) polynucleotide including polymorphism that a 274188377th cytosine base from a first of the 4th chromosome is thymine base and wherein bases of 50 bp before and after the thymine base make up a base sequence having no less than 80% identity to a base sequence of sequence number 2;
(c) polynucleotide including polymorphism that a 274188380th thymine base from a first of the 4th chromosome is guanine base and wherein bases of 50 bp before and after the guanine base make up a base sequence having no less than 80% identity to a base sequence of sequence number 3; and
(d) polynucleotide including polymorphism that a 274188382th adenine base from a first of the 4th chromosome is cytosine base and wherein bases of 50 bp before and after the cytosine base make up a base sequence having no less than 80% identity to a base sequence of sequence number 4.


SEQUENCE LISTING (4 sequences)

SEQ ID NO: 1 - DNA, 101 bp, Lactuca sativa
  source 1..101: mol_type = genomic DNA
ataaaaccataagcagctgcagcataaacaagtgcttgttcatctggagattccccttgataatcaatcaacttctcattgggatcagatgtgtcaacaac

SEQ ID NO: 2 - DNA, 101 bp, Lactuca sativa
  source 1..101: mol_type = genomic DNA
gtcttttgttcaaagcccaaatctgttgttcccttggatatatactaaaattctgaccattttcttctgctttcacaacatgcgaaaagacctccgggggt

SEQ ID NO: 3 - DNA, 101 bp, Lactuca sativa
  source 1..101: mol_type = genomic DNA
ttttgttcaaagcccaaatctgttgttcccttggatatatactaaaactcggaccattttcttctgctttcacaacatgcgaaaagacctccgggggtagt

SEQ ID NO: 4 - DNA, 101 bp, Lactuca sativa
  source 1..101: mol_type = genomic DNA
ttgttcaaagcccaaatctgttgttcccttggatatatactaaaactctgcccattttcttctgctttcacaacatgcgaaaagacctccgggggtagtgg